(19) United States
(12) Patent Application Publication
(10) Pub. No.: US 2024/0124906 A1
(43) Pub. Date: Apr. 18, 2024

(54) METHOD OF PREPARING AN EXTRACT OF CHAYOTE

(71) Applicant: HEBEI RUILONG BIOTECHNOLOGY CO., LTD, Shijiazhuang (CN)

(72) Inventors: Weijia LI, Shijiazhuang (CN); Aimin HE, Shijiazhuang (CN); Lijin LONG, Shijiazhuang (CN)

(73) Assignee: HEBEI RUILONG BIOTECHNOLOGY CO., LTD, Shijiazhuang (CN)

(21) Appl. No.: 18/528,910

(22) Filed: Dec. 5, 2023

Related U.S. Application Data
(63) Continuation of application No. PCT/CN2023/108422, filed on Jul. 20, 2023.

(30) Foreign Application Priority Data
Aug. 2, 2022 (CN) 202210922091.9

(51) Int. Cl. C12P 17/06

(52) U.S. Cl. CPC C12P 17/06

(57) ABSTRACT

The disclosure relates to the technical field of food and medical chemical production, and provides a method of preparing an extract of chayote. The method includes: step 1, adding water and a pretreatment agent of ethoxylated p-tert-octylphenol to a chayote powder to obtain a pretreatment mixture; step 2, adding a stabilizer and a cellulase to the pretreatment mixture to perform an enzymolysis treatment to obtain an enzymatic hydrolysate, and centrifuging the enzymatic hydrolysate to obtain a crude extraction liquid; step 3, concentrating the crude extraction liquid to obtain a concentrate, purifying and drying the concentrate to obtain the extract of chayote.

### CROSS-REFERENCE TO RELATED APPLICATIONS

This application is a continuation of International Application No. PCT/CN2023/108422, filed on Jul. 20, 2023, which claims priority to Chinese Patent Application No. 202210922091.9, filed on Aug. 2, 2022. The disclosures of the aforementioned applications are hereby incorporated by reference in their entireties.

### TECHNICAL FIELD

The disclosure relates to the technical field of food and medical chemical production, and in particular to a method of preparing an extract of chayote.

### BACKGROUND

Chayote belongs to the chayote genus of the Cucurbitaceae family, native to Mexico, Central America and Islands of West India, and introduced to China in 1915. Chayote is planted in Jiangnan area in China, with Yunnan, Guizhou, Zhejiang, Fujian, Guangdong, Sichuan and Taiwan being the most. Chayote is rich in trace elements, and also contains natural functional ingredients such as flavonoids, polysaccharides, pectin, and carotene. It is a nutritious and healthy vegetable that can be used for both medicine and food.

Flavonoids are polyphenols found in flowers, leaves and roots of plants, and have physiological functions such as antioxidation, anti-aging, hypoglycemic effect, hypolipidemic effect and cardiovascular disease prevention. Flavonoids can be used as food additives and raw materials of functional foods, and have a wide range of application prospects.

At present, flavonoids are extracted from chayote mainly by using ethanol. For example, in the method of preparing an extract of chayote of patent application No. CN201610888096.9, the ultrasound extraction was performed by ethanol. However, flavonoids mainly exist in plant cells, and the cell wall of plant cells is mainly composed of cellulose. In order to dissolve flavonoids from plant cells into the solvent, the cell wall of the plant must be destroyed first or the cell wall be decomposed to facilitate the dissolution of flavonoids. Moreover, flavonoids are unstable in the preparation process and are easily oxidized.

### SUMMARY

The present disclosure provides a method of preparing an extract of chayote.

### Technical Problems

The application provides a method of preparing an extract of chayote, so as to solve the problems in the related art that the content of flavonoids is low and the stability of flavonoids is poor in preparing an extract of chayote.

### Technical Solutions

The present disclosure provides a method of preparing an extract of chayote, where the method may include the following step 1 to step 3.

Step 1, adding water and a pretreatment agent to a chayote powder to obtain a pretreatment mixture, where the pretreatment agent is ethoxylated p-tert-octylphenol, and the structural formula of ethoxylated p-tert-octylphenol is as follows:

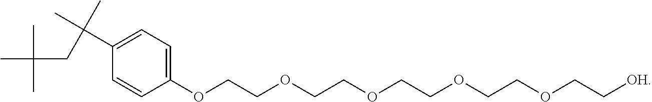 [SMILES: CC(C)(C)CC(C)(C)c1ccc(OCCOCCOCCOCCOCCO)cc1]

Step 2, adding a stabilizer and a cellulase to the pretreatment mixture to perform an enzymolysis treatment to obtain an enzymatic hydrolysate, and centrifuging the enzymatic hydrolysate to obtain a crude extraction liquid.

Step 3, concentrating the crude extraction liquid to obtain a concentrate, purifying and drying the concentrate to obtain the extract of chayote.

As an embodiment of the present application, in step 1, chayote is washed, sliced, dried, crushed, and sieved through 80 mesh to obtain the chayote powder.

As an embodiment of the present application, in step 1, the mass of the water may be about 5 to about 8 times a mass of the chayote powder.

As an embodiment of the present application, the mass of the ethoxylated p-tert-octylphenol may be about 3% to about 5% of the mass of the water.

As an embodiment of the present application, in step 2, the stabilizer comprises ursolic acid and ferulic acid, where the chemical name of the ferulic acid is 3-methoxy-4-hydroxycinnamic acid.

As an embodiment of the present application, in step 2, the ursolic acid and the ferulic acid are diluted with ethanol and added to the pretreatment mixture, a concentration of the ursolic acid in the pretreatment mixture ranging from 0.005 mol/kg to 0.01 mol/kg, and a concentration of the ferulic acid in the pretreatment mixture ranging from 0.01 mol/kg to 0.05 mol/kg.

As an embodiment of the present application, in step 2, a mass of the cellulase may be about 1% of a mass of the pretreatment mixture; where the enzymolysis treatment is conducted at 50° C. for 50 min, and where an enzyme inactivation is performed at 85° C. for 20 min after the enzymolysis treatment.

As an embodiment of the present application, in step 3, the crude extraction liquid is concentrated under reduced pressure at 60° C. till a relative density of the concentrate becomes 1.18 g/cm³, that is, the ratio of the mass (gram (g)) to the volume (centimeter (cm³)) of the concentrate is 1.18.

As an embodiment of the present application, in step 3, the purifying comprises: adsorbing the concentrate by a macroporous adsorption resin column, washing the adsorbed concentrate with distilled water having a volume 5 times the volume of the macroporous adsorption resin column, then eluting the adsorbed concentrate with an ethanol solution with a volume percent concentration of 60% to obtain an eluent; the volume of the 60% ethanol solution may be 4 times the amount of the macroporous adsorption resin column volume; the eluent is collected when eluting the adsorbed concentrate with the 60% ethanol solution not the distilled water.

The packing of the macroporous adsorption resin column used in the present application may be a macromolecule adsorption resin having a macroporous structure, and is a conventional column material in the art. In the process of eluting the concentrate, the elution flow rate is not specifically limited, and can be reasonably adjusted according to the experimental process.

As an embodiment of the present application, in step 3, the eluent obtained in purification is concentrated and then dried.

### Advantageous Effects of the Disclosure

1. In this application, the ethoxylated p-tert-octylphenol is added to the chayote powder for pretreatment before enzymolysis treatment, which can improve the stability and activity of the cellulase, reduce the ineffective adsorption of the cellulase to the chayote powder, and further improve the enzymatic hydrolysis rate and effect of the cellulase. By adopting the enzymolysis method, the permeability of a cell wall can be changed, so that the cell wall becomes more easily broken, expanded and loosened, and the permeability of the cell wall is increased, thereby achieving the purpose of a high extraction rate.
2. Flavonoids are extremely unstable and easily oxidized. In the present application, ursolic acid and ferulic acid are used as stabilizer to protect flavonoids. Due to the addition of ursolic acid and ferulic acid, the thermal stability of flavonoids can be improved during enzymolysis treatment, and thereby the content of flavonoids in the crude extraction liquid is increased. Furthermore, the stability of the extract of chayote during placement can be improved in this application according to the addition of ursolic acid and ferulic acid.
3. In the present application, by adding ethoxylated p-tert-octylphenol as the enzymatic hydrolysis assistant and ursolic acid and ferulic acid as the stabilizer, the extraction rate of flavonoids in the extract of chayote is improved, and the stability of flavonoids during extraction and placement process is also improved.

### DETAILED DESCRIPTION OF ILLUSTRATIVE EMBODIMENTS

The technical solutions in the embodiments of the present application are clearly and completely described with reference to the embodiments of the present application in the following. Apparently, the embodiments to be described are merely a part rather than all of the embodiments of the present application. All other embodiments obtained by a person of ordinary skill in the art based on the embodiments of the present application without creative efforts shall belong to the scope of protection of the present application.

The process of preparing the chayote powder in Embodiment 1 to 3 was: chayote was washed, sliced, dried in an oven at about 60° C., crushed, and sieved through 80 mesh to obtain the chayote powder.

### Embodiment 1

A method of preparing an extract of chayote comprises the following steps:
Step 1, water and ethoxylated p-tert-octylphenol were added to a chayote powder in sequence to obtain a pretreatment mixture. Where a mass of the water was 5 times a mass of the chayote powder, and a mass of the ethoxylated p-tert-octylphenol was 5% of the mass of the water.
Step 2, the ursolic acid and the ferulic acid diluted with ethanol were added to the pretreatment mixture. Where a concentration of the ursolic acid in the pretreatment mixture was 0.01 mol/kg, and a concentration of the ferulic acid in the pretreatment mixture was 0.03 mol/kg. The cellulase with 1% of a mass of the pretreatment mixture was added for an enzymolysis treatment at 50° C. for 50 min, and an enzyme inactivation was performed at 85° C. for 20 min after the enzymolysis treatment to obtain an enzymatic hydrolysate. The enzymatic hydrolysate was centrifuged to obtain a crude extraction liquid.
Step 3, the crude extraction liquid was concentrated under reduced pressure at 60° C. till a relative density of the concentrate was 1.18 g/cm³. The concentrate was adsorbed by a macroporous adsorption resin column, and the adsorbed concentrate was washed with distilled water having a volume 5 times a volume of the macroporous adsorption resin column, then the adsorbed concentrate was eluted with an ethanol solution with a volume percent concentration of 60% to obtain an eluent. The eluent was concentrated and then vacuum dried to obtain the extract of chayote.

### Embodiment 2

A method of preparing an extract of chayote comprises the following steps:
Step 1, water and ethoxylated p-tert-octylphenol were added to a chayote powder in sequence to obtain a pretreatment mixture. Where a mass of the water was 7 times a mass of the chayote powder, and a mass of the ethoxylated p-tert-octylphenol was 4% of the mass of the water.
Step 2, the ursolic acid and the ferulic acid diluted with ethanol were added to the pretreatment mixture. Where a concentration of the ursolic acid in the pretreatment mixture was 0.008 mol/kg, and a concentration of the ferulic acid in the pretreatment mixture was 0.01 mol/kg. The cellulase with 1% of a mass of the pretreatment mixture was added for an enzymolysis treatment at 50° C. for 50 min, and an enzyme inactivation was performed at 85° C. for 20 min after the enzymolysis treatment to obtain an enzymatic hydrolysate. The enzymatic hydrolysate was centrifuged to obtain a crude extraction liquid.
Step 3, the crude extraction liquid was concentrated under reduced pressure at 60° C. till a relative density of the concentrate was 1.18 g/cm³. The concentrate was adsorbed by a macroporous adsorption resin column, and the adsorbed concentrate was washed with distilled water having a volume 5 times a volume of the macroporous adsorption resin column, then the adsorbed concentrate was eluted with an ethanol solution with a volume percent concentration of 60% to obtain an eluent. The eluent was concentrated and then vacuum dried to obtain the extract of chayote.

### Embodiment 3

A method of preparing an extract of chayote comprises the following steps:
Step 1, water and ethoxylated p-tert-octylphenol were added to a chayote powder in sequence to obtain a pretreatment mixture. Where a mass of the water was 8 times a mass of the chayote powder, and a mass of the ethoxylated p-tert-octylphenol was 3% of the mass of the water.
Step 2, the ursolic acid and the ferulic acid diluted with ethanol were added to the pretreatment mixture. Where a concentration of the ursolic acid in the pretreatment mixture was 0.005 mol/kg, and a concentration of the ferulic acid in the pretreatment mixture was 0.05 mol/kg. The cellulase with 1% of a mass of the pretreatment mixture was added for an enzymolysis treatment at 50° C. for 50 min, and an enzyme inactivation was performed at 85° C. for 20 min after the enzymolysis treatment to obtain an enzymatic hydrolysate. The enzymatic hydrolysate was centrifuged to obtain a crude extraction liquid.
Step 3, the crude extraction liquid was concentrated under reduced pressure at 60° C. till a relative density of the concentrate was 1.18 g/cm³. The concentrate was adsorbed by a macroporous adsorption resin column, and the adsorbed concentrate was washed with distilled water having a volume 5 times a volume of the macroporous adsorption resin column, then the adsorbed concentrate was eluted with an ethanol solution with a volume percent concentration of 60% to obtain an eluent. The eluent was concentrated and then vacuum dried to obtain the extract of chayote.

### Embodiment 4

Compared with Embodiment 3, in Embodiment 4, ursolic acid was replaced with an equal amount of ferulic acid, and the others were the same as Embodiment 3.

### Embodiment 5

Compared with Embodiment 1, in Embodiment 5, a mass of the ethoxylated p-tert-octylphenol was 6% of the mass of the water, and the others were the same as Embodiment 1.

### Embodiment 6

Compared with Embodiment 3, in Embodiment 6, a concentration of the ursolic acid in the pretreatment mixture was 0.05 mol/kg, and a concentration of the ferulic acid in the pretreatment mixture was 0.005 mol/kg, and the others were the same as Embodiment 3.

### Comparative Embodiment 1

Compared with Embodiment 3, in Comparative Embodiment 1, ethoxylated p-tert-octylphenol was replaced with an equal amount of tween-80 (polysorbate-80), and the others were the same as Embodiment 3.

### Comparative Embodiment 2

Compared with Embodiment 3, there was no ethoxylated p-tert-octylphenol in Comparative Embodiment 2, and the others were the same as Embodiment 3.

### Comparative Embodiment 3

Compared with Embodiment 3, there were no ursolic acid and ferulic acid in Comparative Embodiment 3, and the others were the same as Embodiment 3.

### Experimental Examples

The extraction rate of flavonoids in crude extraction liquid in Embodiments 1 to 6 and Comparative Embodiments 1 to 3 was determined.
(1) Plotting standard curve of total flavonoids: the standard curve of total flavonoids was plotted according to aluminum nitrate-sodium nitrite colorimetry and rutin as a standard. The rutin standard was dried to constant weight in a drying cabinet at 105° C., 10 milligram (mg) of the dried rutin was accurately weighed and placed in a 100 milliliter (mL) volumetric flask, and the dried rutin was dissolved in 80% ethanol solution to obtain 100 mL of rutin standard solution with a concentration of rutin being 100 milligrams/litre (mg/L). 0, 1.0 mL, 2.0 mL, 3.0 mL, 4.0 mL and 5.0 mL of rutin standard solutions were respectively measured and placed in 10 mL volumetric flasks, and 0.3 mL of 5% sodium nitrite (NaNO₂) solution was respectively added to the 10 mL volumetric flasks to obtain six first solutions. After shaking the six first solutions uniformly and placing them for 6 min, 0.3 mL of 10% aluminum nitrate (Al(NO 3) 3) solution was respectively added to the six first solutions to obtain six second solutions. After shaking the six second solutions uniformly and placing them for 6 min, 4 mL of 4% sodium hydroxide (NaOH) solution was respectively added to the six second solutions to obtain six third solutions. After shaking the six third solutions uniformly and placing them for 20 min, 80% ethanol solution was respectively added to the six third solutions for constant volume and placed for 30 min to obtain six reaction mixtures of rutin standard. The absorbance at a wavelength of 510 nanometers (nm) of the six reaction mixtures of rutin standard was determined by an ultraviolet-visible spectrophotometer. The standard curve was plotted by using absorbances (y) as ordinate and rutin concentrations (x) as abscissa. The results were performed a linear regression to obtain a regression equation of the absorbances and concentrations as y=0.0114x+0.0016, and the correlation coefficient R 2=0.9972.
(2) Determination of flavonoids content in crude extraction liquid of chayote.

A certain amount of crude extraction liquid of chayote was added to a 10 mL volumetric flask, and constant volume was performed by using 80% ethanol solution. The corresponding reagents were added according to the present method in (1) above to prepare sample reaction mixture, while distilled water was as a blank. The absorbance at the wavelength of 510 nm of the sample reaction mixture was determined by the ultraviolet-visible spectrophotometer. The determined absorbance was substituted into the above regression equation y=0.0114x+0.0016 to calculate mass concentration of flavonoids in the crude extraction liquid. The extraction rate of flavonoids was converted by the following formula.

Extraction Rate of Flavonoids (%)=(b×V)/(m×10³)×100%.

In the formula, m indicated the mass of chayote/g; b indicated mass concentration of flavonoids calculated by the regression equation/(mg·L⁻¹); V indicated the extract volume/L.

Table 1 Extraction Rate of Flavonoids in Embodiments 1 to 6 and Comparative Embodiments 1 to 3.

| Items | Extraction Rate of Flavonoids (%) |
|---|---|
| Embodiment 1 | 4.55 |
| Embodiment 2 | 4.43 |
| Embodiment 3 | 4.35 |
| Embodiment 4 | 4.21 |
| Embodiment 5 | 4.25 |
| Embodiment 6 | 4.20 |
| Comparative Embodiment 1 | 4.15 |
| Comparative Embodiment 2 | 3.95 |
| Comparative Embodiment 3 | 3.92 |

It can be seen from Table 1 that Embodiment 1 is the optimal embodiment of the present application, and the extraction rate of flavonoids in the crude extraction liquid of chayote is 4.55%.

Compared with Embodiment 3, in Comparative Embodiment 1, ethoxylated p-tert-octylphenol was replaced with an equal amount of tween-80; there was no ethoxylated p-tert-octylphenol in Comparative Embodiment 2; in Embodiment 4, ursolic acid was replaced with an equal amount of ferulic acid; there were no ursolic acid and ferulic acid in Comparative Embodiment 3; in Embodiment 6, a concentration of the ursolic acid in the pretreatment mixture was 0.05 mol/kg, and a concentration of the ferulic acid in the pretreatment mixture was 0.005 mol/kg. The content of flavonoids in the crude extraction liquid of chayote in Comparative Embodiments 1 to 3 and Embodiments 4 and 6 were less than that in Embodiment 3.

Compared with Embodiment 1, the amount of ethoxylated p-tert-octylphenol was increased in Embodiment 5, but the extraction rate of flavonoids in the crude extraction liquid of chayote was reduced, which may be that increasing the amount of ethoxylated p-tert-octylphenol has an inhibition effect on the cellulase.

The above-mentioned embodiments are only used to illustrate the technical solutions of the disclosure, but not to limit the disclosure; although the present application has been described in detail with reference to the above-mentioned embodiments, those of ordinary skilled in the art should understand that: it is still possible to modify the technical solutions described in the above-mentioned embodiments, or to perform equivalent replacements for some of the technical features. However, these modifications or replacements do not make the essence of the corresponding technical solutions deviate from the spirit and scope of the technical solutions of the embodiments of the present application, and should be included within the protection scope of the present application.

What is claimed is:

1. A method of preparing an extract of chayote (Sechium edule), the method comprising:
step 1, adding water and a pretreatment agent to a chayote powder to obtain a pretreatment mixture, the pretreatment agent being ethoxylated p-tert-octylphenol, and a mass of the ethoxylated p-tert-octylphenol ranging from 3% to 5% of a mass of the water;
step 2, adding a stabilizer and a cellulase to the pretreatment mixture to perform an enzymolysis treatment to obtain an enzymatic hydrolysate, and centrifuging the enzymatic hydrolysate to obtain a crude extraction liquid, the stabilizer comprising ursolic acid and ferulic acid, the ursolic acid and the ferulic acid being diluted with ethanol and added to the pretreatment mixture, a concentration of the ursolic acid in the pretreatment mixture ranging from 0.005 mole/kilogram (mol/kg) to 0.01 mol/kg, and a concentration of the ferulic acid in the pretreatment mixture ranging from 0.01 mol/kg to 0.05 mol/kg; and
step 3, concentrating the crude extraction liquid to obtain a concentrate, purifying and drying the concentrate to obtain the extract of chayote.

2. The method of claim 1, wherein in step 1, chayote is washed, sliced, dried, crushed, and sieved through 80 mesh to obtain the chayote powder.

3. The method of claim 1, wherein in step 1, the mass of the water is 5 to 8 times a mass of the chayote powder.

4. The method of claim 1, wherein in step 2, a mass of the cellulase is 1% of a mass of the pretreatment mixture; wherein the enzymolysis treatment is conducted at 50° C. for 50 minutes (min), and wherein an enzyme inactivation is performed at 85° C. for 20 min after the enzymolysis treatment.

5. The method of claim 1, wherein in step 3, the crude extraction liquid is concentrated under reduced pressure at 60° C. till a relative density of the concentrate is 1.18 gram/centimeter³ (g/cm³).

6. The method of claim 1, wherein in step 3, the purifying comprises: adsorbing the concentrate by a macroporous adsorption resin column, washing the adsorbed concentrate with distilled water having a volume 5 times a volume of the macroporous adsorption resin column, then eluting the adsorbed concentrate with an ethanol solution with a volume percent concentration of 60% to obtain an eluent.
